(19) United States
(12) Patent Application Publication
(10) Pub. No.: US 2008/0153866 A1
(43) Pub. Date: Jun. 26, 2008

(54) Amorphous Taclolimus Solid Dispersion Having an Enhanced Solubility and Pharmaceutical Composition Comprising Same

(75) Inventors: Jong Soo Woo, Gyeonggi-do (KR); Hong Gi Yi, Gyeonggi-do (KR); Jeong Hee Park, Gyeonggi-do (KR)

Correspondence Address: SUGHRUE MION, PLLC, 2100 PENNSYLVANIA AVENUE, N.W., SUITE 800, WASHINGTON, DC, 20037, US

(73) Assignee: Hinmi Pharm Co., Ltd, Hwaseong-gun (KR)

(21) Appl. No.: 11/815,345

(22) PCT Filed: Feb. 3, 2006
(86) PCT No.: PCT/KR2006/000397

§ 371 Date: Aug. 2, 2007

(30) Foreign Application Priority Data
Feb. 4, 2005 (KR) 10-2005-0010302

(51) Int. Cl. A61K 31/4353; A61P 43/00

(52) U.S. Cl. 514/291

(57) ABSTRACT

An amorphous taclolimus solid dispersion comprising taclolimus, a substituted cyclodextrin derivative and an organic acid, which has a high thermodynamic stability and solubility, can provide an enhanced release rate and bioavailability.

### FIELD OF THE INVENTION

The present invention relates to an amorphous taclolimus solid dispersion comprising taclolimus, a substituted cyclodextrin derivative and an organic acid, which exhibits an enhanced bioavailability of taclolimus, and a pharmaceutical composition comprising same.

### BACKGROUND OF THE INVENTION

Taclolimus (or FK-506) of formula (I), (—)-(1R,9S,13R,14S,17R,18E,21S,23S,24R,25S,27R)-17-allyl-1,14-dihydroxy-12-[(E)-2-[(1R,3R,4R)-4-hydroxy-3-methoxycyclohexyl]-1-methylvinyl]-23,25-dimethoxy-13,19,21,27-tetramethyl-11,28-dioxa-4-azatricyclo[22.3.1.0^{4.9}]octacos-18-ene-2,3,10,16-tetrone hydrate [104987-11-3], is a microlide-based immunosuppressive drug discovered by Tanaka and Kuroda et al. (see J. Am. Chem. Soc., 109:5031 (1987) and U.S. Pat. No. 4,894,366).

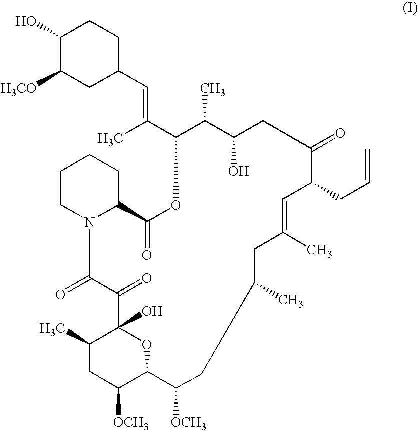

U.S. Food and Drug Administration has approved the use of taclolimus for inhibiting transplantation rejection (Prograf® capsule (Fujisawa, Japan)), and for treating atopic dermatitis (Protopic® as ointment). Further, taclolimus related compounds have proved to be useful for treating diseases such as allergic encephalomyelinitis, collagenous arthritis, obstructive bronchial disease such as asthma, male pattern alopecia, diabetic disease, ophthalmic disease such as posterior uveitis, local anemia related liver damage, glomerulonephritis, systemic lupus erythematosus, multidrug resistance, inflammations of mucous membrane and blood vessel, cytomegalovirus infection, idiopathic thrombocytopenic purpura, and hyperthyroidism.

Taclolimus is a white crystal or crystalline powder, and very soluble in organic solvents such as anhydrous ethanol, but insoluble in water. Thus, when water-insoluble taclolimus is orally administered, its bioavailability is inevitably low.

To overcome this problem, Korean Patent Publication No. 1987-10073 discloses a preparation method of a commercially available Prograf capsule comprising the steps of adding a water-soluble polymer to water-insoluble taclolimus dissolved in an organic solvent; optionally suspending an additive such as an excipient and disintegrant thereto to obtain a homogenous suspension; and removing the organic solvent therefrom in accordance with a conventional method to obtain a solid dispersion composition comprising taclolimus and water-soluble polymer.

The present inventors have unexpectedly discovered that a taclolimus solid dispersion prepared by subjecting taclolimus to spray dryer together with a substituted cyclodextrin derivative and an organic acid exhibits a higher solubility and stability of the active ingredient than any of the existing taclolimus formulations containing hydroxypropyl methylcellulose.

### SUMMARY OF THE INVENTION

Accordingly, it is an object of the present invention to provide a taclolimus solid dispersion which exhibits higher water-solubility and bioavailability than a conventional taclolimus formulation, and a method for the preparation thereof.

It is another object of the present invention to provide a pharmaceutical composition for oral administration comprising the taclolimus solid dispersion.

In accordance with one aspect of the present invention, there is provided an amorphous taclolimus solid dispersion comprising taclolimus, a substituted α-, β- or γ-cyclodextrin derivative of formula (II) and an organic acid:

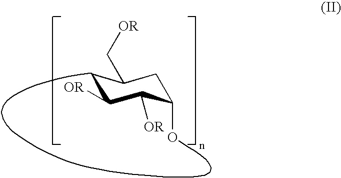

wherein,

n is an integer in the range from 6 to 8; and

R is C₁₋₆alkyl optionally substituted with hydroxyl, carboxy or carboxyC₁₋₄alkoxy, or sulfoC₁₋₄alkoxy.

In accordance with another aspect of the present invention, there is provided the said solid dispersion further comprises a surfactant, a water-soluble polymer or a pharmaceutically acceptable additive.

### BRIEF DESCRIPTION OF DRAWINGS

The above and other objects and features of the present invention will become apparent from the following description of the invention taken in conjunction with the following accompanying drawings, which respectively show:

FIG. 1: Differential scanning calorimeter (DSC) thermograms of crystalline taclolimus (1), the solid dispersion of Example 1 (2) and the mixture of Comparative Example 2 (3);

FIG. 2: Powder X-ray diffraction spectra of the solid dispersion of Example 1 (1), crystalline taclolimus (2) and the mixture of Comparative Example 2 (3);

FIG. 3: Saturated solubility profiles of prograf capsule (control) and the capsules prepared in Preparation Examples 1, 9 and 11, and Comparative Preparation Examples 1 and 2;

FIG. 4: in vitro release profiles of prograf capsule (control) and the capsules prepared in Preparation Example 1 and Comparative Preparation Example 1; and

FIG. 5: in vivo bioavailability profiles of the orally administered prograf capsule (control) and the capsules prepared in Preparation Example 1 and Comparative Preparation Example 1.

### DETAILED DESCRIPTION OF THE INVENTION

Each ingredient of the said solid dispersion comprising taclolimus is described in detail as follows:

### (1) Substituted Cyclodextrin Derivative

The substituted cyclodextrin derivative of formula (II) used in the present invention plays one of the essential roles in the formation of the inventive amorphous solid dispersion having an enhanced water-solubility and bioavailability, and representative examples thereof include 2-hydroxyethyl-β-cyclodextrin, 2-hydroxypropyl-β-cyclodextrin, 2,6-dimethyl-β-cyclodextrin, sulfobutylether-7-β-cyclodextrin, (2-carboxymethoxy)propyl-β-cyclodextrin, 2-hydroxyethyl-γ-cyclodextrin and 2-hydroxypropyl-γ-cyclodextrin, and 2-hydroxyethyl-β-cyclodextrin, 2-hydroxypropyl-β-cyclodextrin, 2,6-dimethyl-β-cyclodextrin and sulfobutylether-7-β-cyclodextrin are preferred. The substituted cyclodextrin derivatives can be used in combination.

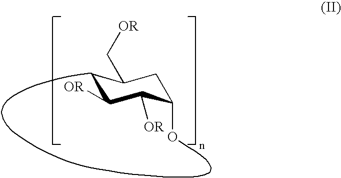

wherein,

n is an integer in the range from 6 to 8; and

R is C₁₋₆alkyl optionally substituted with hydroxyl, carboxy or carboxyC₁₋₄alkoxy, or sulfoC₁₋₄alkoxy.

### (2) Organic Acid

The organic acid of the present invention may be any one of the known pharmaceutically acceptable organic acids, which is used to stabilize taclolimus when the inventive solid dispersion is formulated. Representative examples of the organic acid include erythorbic acid, citric acid, tartaric acid, ascorbic acid, lactic acid, malic acid, succinic acid, acetic acid, trichloroacetic acid, trifluoroacetic acid, dimethyl triamine penta acetic acid, pyruvic acid, malonic acid, myristic acid, picric acid, methanesulfonic acid, ethanesulfonic acid, p-aminobenzoic acid, benzenesulfonic acid, benzoic acid, edetic acid, sorbic acid, adipic acid, gluconic acid, aminocapronic acid, glycyrrhizinic acid, isostearic acid, dodecyl benzenesulfonic acid, fumaric acid, maleic acid, oxalic acid, butyric acid, palmitic acid, sulfonic acid, sulfinic acid, formic acid, propionic acid, tannic acid, pantothenic acid, aspartic acid, aminoacetic acid, DL-a-aminopropionic acid and a mixture thereof, and erythorbic and citric acid are preferred.

### (3) Additive

The additives such as a surfactant, a water-soluble polymer and a pharmaceutically acceptable additive that can be used in the present invention enhance the fluidity and other physical properties of the inventive solid dispersion, and examples thereof include the following.

i) Surfactant

\textcircled{1} polyoxyethylene-sorbitan-fatty acid esters:

mono- or tri-lauric, palmitic, stearic or oleic acid ester (Tween@, Uniquema),

\textcircled{2} sorbitan fatty acid esters:

sorbitan monolauryl, sorbitan monopalmityl or sorbitan monostearyl ester (Span®, Uniquema), \textcircled{3} polyoxyethylene-polyoxypropylene block copolymers (Poloxamers),

\textcircled{4} reaction products of natural or hydrogenated vegetable oil with ethylene glycol:

polyoxyethylene glycolated natural or hydrogenated castor oil (Cremophor®, BASF),

\textcircled{5} D polyoxyethylene fatty acid esters:

polyoxyethylene stearic acid ester (Myrj®, Uniquema),

\textcircled{6} sodium dioctyl sulfosuccinate or sodium lauryl sulfate, and

\textcircled{7} mixture of glycerol mono-, di- and tri-ester, polyethylene glycol mono- and di-ester or polyethylene glycols (Gelucire®, Gattefosse)

Among the above-mentioned surfactants, polyoxyethylene-polyoxypropylene block copolymer, polyoxyethylene fatty acid esters, a mixture of glycerol mono-, di- and tri-ester, polyethylene glycol mono- and di-ester, polyethylene glycols, sodium dioctyl sulfosuccinate and sodium lauryl sulfate are preferred, and polyoxyethylene-polyoxypropylene block copolymer is more preferred.

ii) Water-Soluble Polymer

In the present invention, one or more water-soluble polymers can optionally be added to improve the fluidity and other physical properties of the solid dispersion. The water-soluble polymer can be selected from the group consisting of alkyl cellulose such as methyl cellulose; hydroxyalkyl cellulose such as hydroxymethyl cellulose, hydroxyethyl cellulose, hydroxypropyl cellulose and hydroxybutyl cellulose; hydroxyalkylalkyl cellulose such as hydroxyethylmethyl cellulose and hydroxypropylmethyl cellulose; carboxyalkyl cellulose such as carboxymethyl cellulose; alkali metal of carboxyalkyl cellulose such as sodium carboxymethyl cellulose; carboxyalkylalkyl cellulose such as carboxymethylethyl cellulose; carboxyalkyl cellulose ester; starch; pectin such as sodium carboxymethyl amylopectin; chitin derivative such as chitosan; polysaccharides such as alginic acid, alkali metal and ammonium salt thereof, caragenan, galactomannan, tragacanth, agar-agar, arabic gum, guar gum and xanthan gum; polymetacrylic acid and salt thereof; polymetacrylic acid and salt thereof, metacrylate copolymer, aminoalkyl metacrylate copolymer; polyvinyl acetal and diethyl aminoacetate; saccharic type surfactant such as sucrose distearate, sucrose mono/distearate and sucrose monopalmitate; polyvinyl alcohol; polyvinyl pyrrolidone and polyvinyl pyrrolidone-vinyl acetate copolymer; polyalkylene oxide such as polyethylene oxide and polypropylene oxide; and ethylene oxide-propylene oxide copolymer.

Among the above-mentioned water-soluble polymers, alkyl cellulose, hydroxyalkyl cellulose, hydroxyalkylalkyl cellulose and polyvinyl pyrrolidone are preferred, and hydroxypropylmethyl cellulose, hydroxypropyl cellulose and polyvinyl pyrrolidone are more preferred.

iii) Pharmaceutically Acceptable Additive

In the present invention, one or more pharmaceutically acceptable excipients can optionally be added to improve the fluidity and other physical properties of the solid dispersion in the preparation of an oral administration composition comprising the solid dispersion. The pharmaceutically acceptable excipient can be selected from the group consisting of lactose, starch, sodium starch glycolate, crospovidone, croscarmellose sodium, maltodextrin, microcrystalline cellulose, calcium phosphate, calcium bicarbonate and crystalline cellulose. Further, lubricants such as stearic acid, magnesium stearate and talc can be used in the present invention.

In the preparation of the inventive amorphous taclolimus solid dispersion, taclolimus, the substituted cyclodextrin derivative and the organic acid as a pharmaceutical active ingredient may be used in amounts corresponding to a weight ratio in the range of 1:0.1˜20:0.1˜10, preferably 1:0.1˜10:0.1˜5. The surfactant or the pharmaceutically acceptable additive may be used in an amount of 20 and less weight ratio based on taclolimus. In addition, the compositions described in Examples of the present invention can be referred to preferable exemplify the effects of the present invention.

The amorphous taclolimus solid dispersion of the present invention does not exhibit any endothermic peak in its DSC scan nor a crystalline refractive peak in its powder X-ray diffraction spectrum, demonstrating that taclolimus contained in the inventive dispersion is of a stable amorphous form.

The inventive solid dispersion can be prepared by a method comprising the steps of (a) dispersing or dissolving the substituted cyclodextrin derivative and organic acid in an organic solvent; (b) dissolving taclolimus in an organic solvent; and (c) mixing the dispersion and solution obtained above, followed by removing the solvent therefrom.

In step (a), one or more surfactants and pharmaceutically acceptable additives can optionally be dissolved or dispersed in the solution. The organic solvent which may be used in step (b) is ethanol, isopropyl alcohol, acetone, acetonitrile, dichloromethane, chloroform or any of the suitable organic solvent. In step (c), the solvent can be removed by spray drying, roller drying, solvent precipitation or freeze drying to obtain an amorphous taclolimus solid dispersion.

Further, the present invention provides a pharmaceutical composition of taclolimus for oral administration comprising the solid dispersion together with a pharmaceutically acceptable carrier, excipient and additive. The pharmaceutical composition can be formulated in the form of powder, granule, tablet, soft or hard capsule, pill, or coated formulation in accordance with any of the conventional methods. For example, the solid dispersion may be filled into a hard capsule in the form of powder or granule together with a lubricant or other pharmaceutical additives, or made in the form of tablet together with a pharmaceutical additive for tabletting and then optionally coated in accordance with any of the conventional methods to obtain a coated formulation.

The inventive formulation can be administered orally in a typical amount in a single dose or in divided doses.

The following Examples are intended to further illustrate the present invention without limiting its scope.

### EXAMPLES

### Preparation of Taclolimus Solid Dispersion

### Example 1

400 mg of 2-hydroxypropyl-β-cyclodextrin (Aldrich, USA), 20 mg of citric acid and 20 mg of poloxamer 188 (Lutrol F68, BASF) were added to a mixture of MC (methylene chloride) and ethanol and stirred until the solution became clear. Then, 400 mg of lactose was added thereto and dispersed homogeneously. 100 mg of taclolimus dissolved in ethanol was mixed with the dispersion, and subjected to spray drying while maintaining the entrance and exit temperature of the spray dryer (Mini spray dryer B-191, Buchi, Switzerland), at 60° C. and 45-50° C., respectively, to obtain a taclolimus solid dispersion having the components listed in Table 1.

### Example 2

A taclolimus solid dispersion having the components listed in Table 1 was prepared by repeating the procedure of Example 1 except for using 200 mg of 2-hydroxypropyl-β-cyclodextrin.

### Example 3

A taclolimus solid dispersion having the components listed in Table 1 was prepared by repeating the procedure of Example 1 except for using 800 mg of 2-hydroxypropyl-β-cyclodextrin.

TABLE 1

| | Example 1 | Example 2 | Example 3 |
|---|---|---|---|
| Ingredients | mg/formulation | mg/formulation | mg/formulation |
| Taclolimus | 100 | 100 | 100 |
| 2-Hydroxypropyl- | 400 | 200 | 800 |
| β-cyclodextrin | | | |
| Citric acid | 20 | 20 | 20 |
| Lactose | 400 | 400 | 400 |
| Poloxamer 188 | 20 | 20 | 20 |

### Example 4

A taclolimus solid dispersion having the components listed in Table 2 was prepared by repeating the procedure of Example 1 except for using 2,6-dimethyl-β-cyclodextrin (CAVASOL® W7 M Pharma, WAKER) instead of 2-hydroxypropyl-β-cyclodextrin.

### Example 5

A taclolimus solid dispersion having the components listed in Table 2 was prepared by repeating the procedure of Example 1 except for using sulfobutylether-7-β-cyclodextrin (CAPTISOL®, WAKER) instead of 2-hydroxypropyl-β-cyclodextrin.

### Example 6

A taclolimus solid dispersion having the components listed in Table 2 was prepared by repeating the procedure of Example 1 except for using erythorbic acid instead of citric acid as an organic acid.

TABLE 2

| | Example 4 | Example 5 | Example 6 |
|---|---|---|---|
| Ingredients | mg/formulation | mg/formulation | mg/formulation |
| Taclolimus | 100 | 100 | 100 |
| 2-Hydroxypropyl- | — | — | 400 |
| β-cyclodextrin | | | |
| 2,6-Dimethyl- | 400 | — | — |
| β-cyclodextrin | | | |
| Sulfobutylether-7- | — | 400 | — |
| β-cyclodextrin | | | |
| Citric acid | 20 | 20 | — |
| Erythorbic acid | — | — | 20 |
| Lactose | 400 | 400 | 400 |
| Poloxamer 188 | 20 | 20 | 20 |

### Example 7

A taclolimus solid dispersion having the components listed in Table 3 was prepared by repeating the procedure of Example 1 except for using Myrj 52S (Uniqema) instead of poloxamer 188 as a surfactant.

### Example 8

A taclolimus solid dispersion having the components listed in Table 3 was prepared by repeating the procedure of Example 1 except for using sodium lauryl sulfate instead of poloxamer 188 as a surfactant.

### Example 9

A taclolimus solid dispersion having the components listed in Table 3 was prepared by repeating the procedure of Example 1 except for using solutol instead of poloxamer 188 as a surfactant.

TABLE 3

| | Example 7 | Example 8 | Example 9 |
|---|---|---|---|
| Ingredients | mg/formulation | mg/formulation | mg/formulation |
| Taclolimus | 100 | 100 | 100 |
| 2-Hydroxypropyl- | 400 | 400 | 400 |
| β-cyclodextrin | | | |
| Citric acid | 20 | 20 | 20 |
| Lactose | 400 | 400 | 400 |
| Myrj 52S | 20 | — | — |
| Sodium lauryl sulfate | — | 20 | — |
| Solutol | — | — | 20 |

### Example 10

A taclolimus solid dispersion having the components listed in Table 4 was prepared by repeating the procedure of Example 1 except for using starch instead of lactose as a pharmaceutical additive.

### Example 11

A taclolimus solid dispersion having the components listed in Table 4 was prepared by repeating the procedure of Example 1 except for using maltodextrin instead of lactose as a pharmaceutical additive.

### Example 12

A taclolimus solid dispersion having the components listed in Table 4 was prepared by repeating the procedure of Example 1 except for using microcrystalline cellulose instead of lactose as a pharmaceutical additive.

TABLE 4

| | Example 10 | Example 11 | Example 12 |
|---|---|---|---|
| Ingredients | mg/formulation | mg/formulation | mg/formulation |
| Taclolimus | 100 | 100 | 100 |
| 2-Hydroxypropyl- | 400 | 400 | 400 |
| β-cyclodextrin | | | |
| Citric acid | 20 | 20 | 20 |
| Starch | 400 | — | — |
| Maltodextrin | — | 400 | — |
| Microcrystalline | — | — | 400 |
| cellulose | | | |
| Poloxamer 188 | 20 | 20 | 20 |

### Example 13

A taclolimus solid dispersion having the components listed in Table 5 was prepared by repeating the procedure of Example 1 except for not using poloxamer 188.

### Example 14

A taclolimus solid dispersion having the components listed in Table 5 was prepared by repeating the procedure of Example 1 except for not using lactose.

### Example 15

A taclolimus solid dispersion having the components listed in Table 5 was prepared by repeating the procedure of Example 1 except for not using poloxamer 188 and lactose.

TABLE 5

| | Example 13 | Example 14 | Example 15 |
|---|---|---|---|
| Ingredients | mg/formulation | mg/formulation | mg/formulation |
| Taclolimus | 100 | 100 | 100 |
| 2-Hydroxypropyl-β- | 400 | 400 | 400 |
| cyclodextrin | | | |
| Citric acid | 20 | 20 | 20 |
| Lactose | 400 | — | — |
| Poloxamer 188 | — | 20 | — |

### Example 16

A taclolimus solid dispersion having the components listed in Table 6 was prepared by repeating the procedure of Example 8 except for using 10 mg of citric acid.

### Example 17

A taclolimus solid dispersion having the components listed in Table 6 was prepared by repeating the procedure of Example 8 except for using 50 mg of citric acid.

### Example 18

A taclolimus solid dispersion having the components listed in Table 6 was prepared by repeating the procedure of Example 8 except for using 100 mg of citric acid.

TABLE 6

| | Example 16 | Example 17 | Example 18 |
|---|---|---|---|
| Ingredients | mg/formulation | mg/formulation | mg/formulation |
| Taclolimus | 100 | 100 | 100 |
| 2-Hydroxypropyl-β- | 400 | 400 | 400 |
| cyclodextrin | | | |
| Citric acid | 10 | 50 | 100 |
| Lactose | 400 | 400 | 400 |
| Sodium lauryl sulfate | 20 | 20 | 20 |

### Example 19

A taclolimus solid dispersion having the components listed in Table 7 was prepared by repeating the procedure of Example 1 except for using 800 mg of lactose.

### Example 20

A taclolimus solid dispersion having the components listed in Table 7 was prepared by repeating the procedure of Example 1 except for using 1,500 mg of lactose.

### Example 21

A taclolimus solid dispersion having the components listed in Table 7 was prepared by repeating the procedure of Example 1 except for using 2,000 mg of lactose.

TABLE 7

| | Example 19 | Example 20 | Example 21 |
|---|---|---|---|
| Ingredients | mg/formulation | mg/formulation | mg/formulation |
| Taclolimus | 100 | 100 | 100 |
| 2-Hydroxypropyl-β- | 400 | 400 | 400 |
| cyclodextrin | | | |
| Citric acid | 20 | 20 | 20 |
| Lactose | 800 | 1,500 | 2,000 |
| Poloxamer 188 | 20 | 20 | 20 |

### Example 22

A taclolimus solid dispersion having the components listed in Table 8 was prepared by repeating the procedure of Example 1 except for using 10 mg each of citric acid and erythorbic acid as an organic acid.

### Example 23

A taclolimus solid dispersion having the components listed in Table 8 was prepared by repeating the procedure of Example 1 except for using 200 mg each of lactose and microcrystalline cellulose as a pharmaceutical additive.

### Example 24

A taclolimus solid dispersion having the components listed in Table 8 was prepared by repeating the procedure of Example 1 except for using 10 mg each of poloxamer 188 and sodium lauryl sulfate as a surfactant.

TABLE 8

| | Example 22 | Example 23 | Example 24 |
|---|---|---|---|
| Ingredients | mg/formulation | mg/formulation | mg/formulation |
| Taclolimus | 100 | 100 | 100 |
| 2-Hydroxypropyl-β- | 400 | 400 | 400 |
| cyclodextrin | | | |
| Citric acid | 10 | 20 | 20 |
| Erythorbic acid | 10 | — | — |
| Lactose | 400 | 200 | 400 |
| Microcrystalline | — | 200 | — |
| cellulose | | | |
| Poloxamer 188 | 20 | 20 | 10 |
| Sodium lauryl sulfate | — | — | 10 |

### Example 25

A taclolimus solid dispersion having the components listed in Table 9 was prepared by repeating the procedure of Example 1 except for using 200 mg each of 2-hydroxypropyl-β-cyclodextrin and sulfobutylether-7-β-cyclodextrin.

### Example 26

A taclolimus solid dispersion having the components listed in Table 9 was prepared by repeating the procedure of Example 1 except for using 200 mg of lactose and 200 mg of additional hydroxypropylmethyl cellulose 2910 as a water-soluble polymer.

### Example 27

A taclolimus solid dispersion having the components listed in Table 9 was prepared by repeating the procedure of Example 1 except for using 200 mg of lactose and 200 mg of additional polyvinyl pyrrolidone as a water-soluble polymer.

TABLE 9

| | Example 25 | Example 26 | Example 27 |
|---|---|---|---|
| Ingredients | mg/formulation | mg/formulation | mg/formulation |
| Taclolimus | 100 | 100 | 100 |
| 2-Hydroxypropyl-β- | 200 | 400 | 400 |
| cyclodextrin | | | |
| Sulfobutylether-7-β- | 200 | — | — |
| cyclodextrin | | | |
| Citric acid | 20 | 20 | 20 |
| Hydroxypropylmethyl | — | 200 | — |
| cellulose 2910 | | | |
| Polyvinyl pyrrolidone | — | — | 200 |
| Lactose | 400 | 200 | 200 |
| Poloxamer 188 | 20 | 20 | 20 |

### Comparative Example 1

The same components of Example 1 (see Table 10) were simply mixed without spry drying to obtain a mixture.

### Comparative Example 2

A taclolimus solid dispersion having the components listed in Table 10 was prepared by repeating the procedure of Example 1 except for not using 2-hydroxypropyl-β-cyclodextrin.

### Comparative Example 3

A taclolimus solid dispersion having the components listed in Table 10 was prepared by repeating the procedure of Example 1 except for not using citric acid.

TABLE 10

| | Comparative | Comparative | Comparative |
|---|---|---|---|
| | Example 1 | Example 2 | Example 3 |
| Ingredients | mg/formulation | mg/formulation | mg/formulation |
| Taclolimus | 100 | 100 | 100 |
| 2-Hydroxypropyl-β- | 400 | — | 400 |
| cyclodextrin | | | |
| Citric acid | 20 | 20 | — |
| Lactose | 400 | 400 | 400 |
| Poloxamer 188 | 20 | 20 | 20 |

### Preparation Examples

### Preparation of Capsule Formulation

### Preparation Example 1

940 mg of the solid dispersion of Example 1 (taclolimus 100 mg), 5,495 mg of lactose and 65 mg of magnesium stearate were mixed homogeneously, and the resulting mixture, in an amount corresponding to 1 mg of taclolimus, was filled into a gelatin capsule No. 5 to obtain a capsule formulation having the components listed in Table 11.

### Preparation Example 2

A capsule formulation having the components listed in Table 11 was prepared by repeating the procedure of Preparation Example 1 except for using 1,340 mg of the solid dispersion of Example 3 (taclolimus 100 mg) and 5,095 mg of lactose.

### Preparation Example 3

A capsule formulation having the components listed in Table 11 was prepared by repeating the procedure of Preparation Example 1 except for using 940 mg of the solid dispersion of Example 5 (taclolimus 100 mg).

TABLE 11

| | Preparation | Preparation | Preparation |
|---|---|---|---|
| | Example 1 | Example 2 | Example 3 |
| Ingredients | mg/formulation | mg/formulation | mg/formulation |
| Solid dispersion | 940 | — | — |
| of Example 1 | | | |
| (taclolimus: 1 mg) | | | |
| Solid dispersion | — | 1,340 | — |
| of Example 3 | | | |
| (taclolimus: 1 mg) | | | |
| Solid dispersion | — | — | 940 |
| of Example 5 | | | |
| (taclolimus: 1 mg) | | | |
| Lactose | 5,495 | 5,095 | 5,495 |
| Magnesium stearate | 65 | 65 | 65 |
| Total | 6,500 | 6,500 | 6,500 |

### Preparation Example 4

A capsule formulation having the components listed in Table 12 was prepared by repeating the procedure of Preparation Example 1 except for using 940 mg of the solid dispersion of Example 6 (taclolimus 100 mg).

### Preparation Example 5

A capsule formulation having the components listed in Table 12 was prepared by repeating the procedure of Preparation Example 1 except for using 940 mg of the solid dispersion of Example 8 (taclolimus 100 mg).

### Preparation Example 6

A capsule formulation having the components listed in Table 12 was prepared by repeating the procedure of Preparation Example 1 except for using 940 mg of the solid dispersion of Example 11 (taclolimus 100 mg).

TABLE 12

| | Preparation | Preparation | Preparation |
|---|---|---|---|
| | Example 4 | Example 5 | Example 6 |
| Ingredients | mg/formulation | mg/formulation | mg/formulation |
| Solid dispersion of | 940 | — | — |
| Example 6 | | | |
| (taclolimus: 1 mg) | | | |
| Solid dispersion of | — | 940 | — |
| Example 8 | | | |
| (taclolimus: 1 mg) | | | |
| Solid dispersion of | — | — | 940 |
| Example 11 | | | |
| (taclolimus: 1 mg) | | | |
| Lactose | 5,495 | 5,095 | 5,495 |
| Magnesium stearate | 65 | 65 | 65 |
| Total | 6,500 | 6,500 | 6,500 |

### Preparation Example 7

A capsule formulation having the components listed in Table 13 was prepared by repeating the procedure of Preparation Example 1 except for using 920 mg of the solid dispersion of Example 13 (taclolimus 100 mg) and 5,515 mg of lactose.

### Preparation Example 8

A capsule formulation having the components listed in Table 13 was prepared by repeating the procedure of Preparation Example 1 except for using 540 mg of the solid dispersion of Example 14 (taclolimus 100 mg) and 5,895 mg of lactose.

### Preparation Example 9

A capsule formulation having the components listed in Table 13 was prepared by repeating the procedure of Preparation Example 1 except for using 520 mg of the solid dispersion of Example 15 (taclolimus 100 mg) and 5,915 mg of lactose.

TABLE 13

| | Preparation | Preparation | Preparation |
|---|---|---|---|
| | Example 7 | Example 8 | Example 9 |
| Ingredients | mg/formulation | mg/formulation | mg/formulation |
| Solid dispersion of | 940 | — | — |
| Example 13 | | | |
| (taclolimus: 1 mg) | | | |
| Solid dispersion of | — | 540 | — |
| Example 14 | | | |
| (taclolimus: 1 mg) | | | |
| Solid dispersion of | — | — | 520 |
| Example 15 | | | |
| (taclolimus: 1 mg) | | | |
| Lactose | 5,515 | 5,895 | 5,915 |
| Magnesium stearate | 65 | 65 | 65 |
| Total | 6,500 | 6,500 | 6,500 |

### Preparation Example 10

A capsule formulation having the components listed in Table 14 was prepared by repeating the procedure of Preparation Example 1 except for using 320 mg of the solid dispersion of Example 15 (taclolimus 100 mg) and 6,115 mg of lactose.

### Preparation Example 11

A capsule formulation having the components listed in Table 14 was prepared by repeating the procedure of Preparation Example 1 except for using microcrystalline cellulose instead of lactose as a pharmaceutical additive.

### Preparation Example 12

A capsule formulation having the components listed in Table 14 was prepared by repeating the procedure of Preparation Example 1 except for using 5,195 mg of lactose and 300 mg of croscarmellose sodium as a pharmaceutical additive.

TABLE 14

| | Preparation | Preparation | Preparation |
|---|---|---|---|
| | Example 10 | Example 11 | Example 12 |
| Ingredients | mg/formulation | mg/formulation | mg/formulation |
| Solid dispersion of | 320 | — | — |
| Example 15 | | | |
| (taclolimus: 1 mg) | | | |
| Solid dispersion of | — | 940 | 940 |
| Example 1 | | | |
| (taclolimus: 1 mg) | | | |
| Lactose | 6,115 | — | 5,195 |
| Microcrystalline | — | 5,495 | — |
| cellulose | | | |
| Croscarmellose | — | — | 300 |
| sodium | | | |
| Magnesium stearate | 65 | 65 | 65 |
| Total | 6,500 | 6,500 | 6,500 |

### Preparation Example 13

A capsule formulation having the components listed in Table 15 was prepared by repeating the procedure of Preparation Example 1 except for using 4,845 mg of lactose and 650 mg of additional hydroxypropylmethyl cellulose as a water-soluble polymer.

### Preparation Example 14

A capsule formulation having the components listed in Table 15 was prepared by repeating the procedure of Preparation Example 1 except for using 4,845 mg of lactose and 650 mg of crospovidone as a pharmaceutical additive.

### Preparation Example 15

A capsule formulation having the components listed in Table 15 was prepared by repeating the procedure of Preparation Example 1 except for using 4,845 mg of calcium phosphate and 650 mg of crospovidone instead of lactose.

TABLE 15

| | Preparation | Preparation | Preparation |
|---|---|---|---|
| | Example 13 | Example 14 | Example 15 |
| Ingredients | mg/formulation | mg/formulation | mg/formulation |
| Solid dispersion of | 940 | 940 | 940 |
| Example 1 | | | |
| (taclolimus: 1 mg) | | | |
| Lactose | 4,845 | 4,845 | — |
| Calcium phosphate | — | — | 4,845 |
| Hydroxypropylmethyl | 650 | — | — |
| cellulose | | | |
| Crospovidone | — | 650 | 650 |
| Magnesium stearate | 65 | 65 | 65 |
| Total | 6,500 | 6,500 | 6,500 |

### Comparative Preparation Example 1

A capsule formulation having the components listed in Table 16 was prepared by repeating the procedure of Preparation Example 1 except for using 940 mg of the mixture of Comparative Example 1 (taclolimus 100 mg).

### Comparative Preparation Example 2

A capsule formulation having the components listed in Table 16 was prepared by repeating the procedure of Preparation Example 1 except for using 540 mg of the solid dispersion of Comparative Example 2 (taclolimus 100 mg) and 5,895 mg of lactose.

### Comparative Preparation Example 3

A capsule formulation having the components listed in Table 16 was prepared by repeating the procedure of Preparation Example 1 except for using 920 mg of the solid dispersion of Comparative Example 3 (taclolimus 100 mg) and 5,515 mg of lactose.

TABLE 16

| | Comparative | Comparative | Comparative |
|---|---|---|---|
| | Preparation | Preparation | Preparation |
| | Example 1 | Example 2 | Example 3 |
| Ingredients | mg/formulation | mg/formulation | mg/formulation |
| Mixture of | 940 | — | — |
| Comparative | | | |
| Example 1 | | | |
| (taclolimus: 1 mg) | | | |
| Solid dispersion of | — | 540 | — |
| Comparative | | | |
| Example 2 | | | |
| (taclolimus: 1 mg) | | | |
| Solid dispersion of | — | — | 920 |
| Comparative | | | |
| Example 3 | | | |
| (taclolimus: 1 mg) | | | |
| Lactose | 5,495 | 5,895 | 5,515 |
| Magnesium stearate | 65 | 65 | 65 |
| Total | 6,500 | 6,500 | 6,500 |

### Test Example 1

### Thermodynamic Stability Test

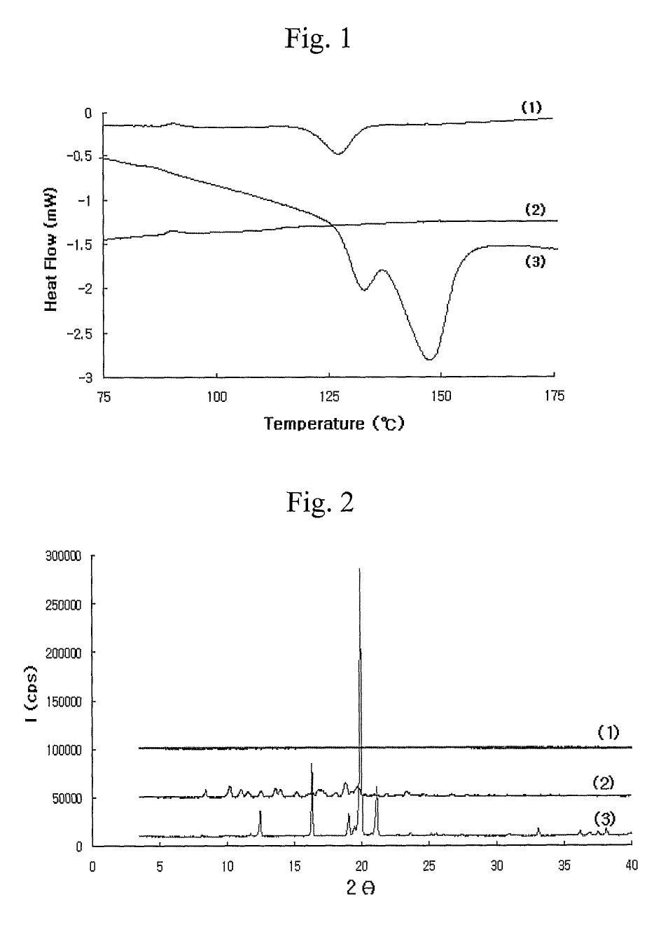

The mixture of Comparative Example 1, solid dispersion of Example 1 and crystalline taclolimus were each subjected to a thermodynamic stability test by examining the change in the endothermic peak in its DSC scan. The temperatures representing respective endothermic peaks are represented in Table 17, and the overall DSC results are shown in FIG. 1.

TABLE 17

| | Endothermic peak temperature (° C.) |
|---|---|
| Comparative Example 1 | 127.9 |
| Example 1 | None |
| Crystalline taclolimus | 127.0 |

As can be seen in Table 17 and FIG. 1, the taclolimus solid dispersion of the present invention did not show any distinctive endothermic peak in the DSC scan thereof, and therefore, it exists in an amorphous form which is stable in the temperature range examined.

### Test Example 2

### X-ray powder diffraction analysis

X-ray powder diffraction patterns of crystalline taclolimus, the mixture of Comparative Example 1 and solid dispersion of Example 1 were determined by using M18XHF-SRA (Macscience Co., LTD, Japan) under the conditions of Cu X-ray, 40 kV and 300 mA, and the results are shown in FIG. 2.

As can be seen in FIG. 2, the taclolimus in the solid dispersion of the present invention became amorphous and thermodynamically stable by the process of spray drying.

### Test Example 3

### Solubility Test

The formulations of Preparation Examples of 1, 9 and 11, as well as Comparative Preparation Examples 1 and 2 were each subjected to a solubility test using Prograf capsule as a control. A sample containing 5 mg of taclolimus taken from each formulation was dissolved in 10 ml of distilled water, and stored in a 25° C. water bath. Test samples were collected at 1, 3, 6, and 24 hours after storage, and the amount of taclolimus in each sample was measured under the following conditions:

[Test Method: Liquid Chromatography]

Column—TSK gel ODS 80 TM column (150 mm×4.6 mm, 5 μm),

Mobile phase—water:isopropanol:tetrahydrofuran=5:2:2 (v/v/v),

Injection volume—20 μl,

Flow rate—control holding time of taclolimus to be about 10 min,

Column temperature—50° C., and

Detection—220 nm.

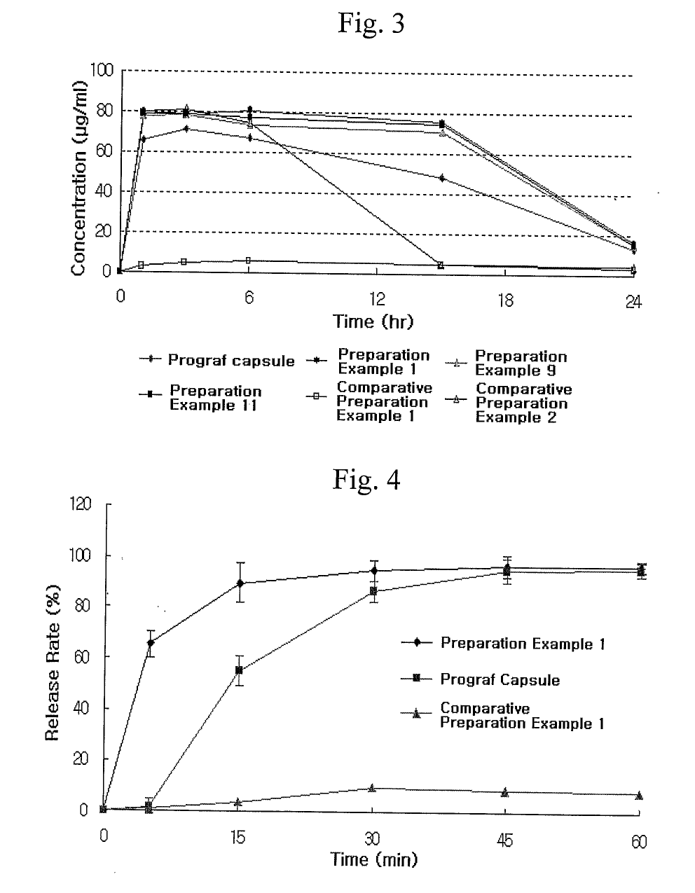

The results are shown in FIG. 3.

As can be seen in FIG. 3, each of the inventive solid dispersion of taclolimus had a markedly higher solubility than the simple mixture of Comparative Preparation Example 1. Further, each formulation comprising the solid dispersion using the cyclodextrin derivative maintained a higher solubility for 15 hours than either the formulation of Comparative Preparation Example 2 or the control formulation when stored in a 25° C. water bath for 24 hours. Therefore, the use of substituted cyclodextrin derivative inhibits the crystallization of the drug in an aqueous solution and remains in the amorphous and bioavailable form over a long period of time.

### Test Example 4

### In Vitro Release Test

The formulations of Preparation Example 1 and Comparative Preparation Example 1 were each subjected to an in vitro release-test in accordance with the release-test method described in Korea pharmacopoeia (the paddle method) using Prograf capsule (1 mg, Fujisawa island) as a control formulation, and the release pattern was analyzed under the following conditions:

[Release-Test Method]
Release-test system—Erweka DT 80,
Release solution—900 ml of distilled water degassed for 10 mins under a reduced pressure,
Temperature of release solution—37±0.5° C.,
Rotation speed—100 rpm, and
Sample amount—1 capsule per each sinker.

[Analysis Method: Liquid Chromatography]

Column—Inertsil CN-3 (150 mm×4.6 mm, 5 μm),

Mobile phase—water:acetonitrile:isopropanol=7:2:1 (v/v/v),

Injection volume—300 μl,

Flow rate—0.5 ml/min,

Column temperature—50° C., and

Detection—210 nm.

The results are shown in FIG. 4.

As can be seen in FIG. 4, the formulation comprising the amorphous taclolimus solid dispersion of the present invention (Formulation 1) exhibited a release rate which was similar to that of the control, but markedly higher than that of the formulation of taclolimus mixed with other components (Comparative Formulation 1).

### Test Example 5

### Stability Test

The formulations of Preparation Example 1 and Comparative Preparation Example 3 were stored under an accelerated aging condition at 60° C. together with 1 mg of Prograf (hard capsule, Fujisawa island) as a control, and the time-dependent changes in the production of impurities and tautomeric isomers thereof were analyzed by HPLC under the following conditions:

Column—2 Supelcosil LC-Diol (250 mm×4 mm, 5 μm) were connected longitudinally,

Mobile phase—n-hexane:n-butylchloride:acetonitrile=7:2:1 (v/v/v),

Injection volume—20 μl,

Flow rate—control holding time of taclolimus to be about 15 min,

Column temperature—25° C., and

Detection—225 nm.

The results are shown in Table 18.

TABLE 18

| | | Impurity | Totomeric | |
|---|---|---|---|---|
| | | (%) | material (%) | |
| | | Unknown | Tautomeric | Tautomeric |
| | | peak | material I | material II |
| Prograf capsule | Initial | 0.08 | 0.23 | 0.81 |
| | 4 weeks | 1.26 | 0.68 | 4.30 |
| Comparative | Initial | 0.00 | 0.19 | 0.67 |
| preparation Example 3 | 4 weeks | 1.20 | 0.64 | 3.35 |
| Preparation Example 1 | Initial | 0.00 | 0.05 | 0.09 |
| | 4 weeks | 0.96 | 0.28 | 0.29 |

As can be seen in Table 18, taclolimus in the inventive formulation comprising a substituted cyclodextrin derivative and an organic acid (Preparation Example 1) was far more stable and resistant to the production of tautomeric materials or impurities than the control or the formulation of Comparative Preparation Example 3 under the severe aging condition.

### Test Example 6

### In Vivo Absorption Test

The formulations of Preparation Example 1 and Comparative Preparation Example 1 were each subjected to an in vivo absorption test using a control to examine the bioavailability of the orally administered inventive compounds.

5 male Sprague-Dawley rats (weight: 250 g each; 14-15 week old) allocated for each formulation were acclimated for more than 4 days, while allowing free access to feed and water. The rats were then put on a 48-hour fast, while they were allowed to free access to water.

The rats were orally administered with the test or control formulations, in an amount corresponding to 10 mg/kg of taclolimus together with water. Blood samples were taken from the rats before the administration, and 0.5, 1, 1.5, 2, 3, 4, 5, 7 and 24 hours after the administration.

400 μl of a mixture of 0.2 M ZnSO₄ and MeOH (2:8 (v/v)) was added to 200 μl of each blood sample, and the mixture was shaken. The mixture was centrifuged at 12,000 rpm for 30 seconds to obtain a supernatant, which was filtered through a 0.22 μm filter, and analyzed by LC-MS under the following conditions:

Column—Waters MS C18 (150 mm×2.1 mm with guard column),

Mobile phase—concentration gradient (65% MeOH≦95% MeOH),

Injection volume—301,

Flow rate—0.3 ml/min, and

Detection—SIR mode m/z: 826.7 (Na adduct).

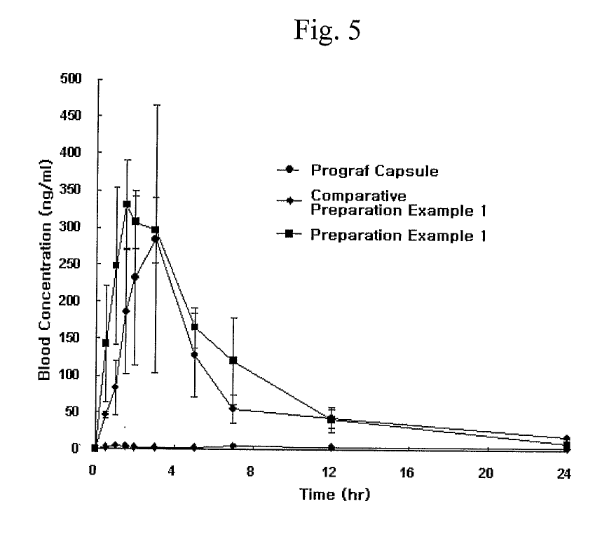

The results are shown in Table 19 and FIG. 5.

TABLE 19

| | AUC*¹ (ng · hr/ml) | Cₘₐₓ*² (ng/ml) | Tₘₐₓ*³ (hr) |
|---|---|---|---|
| Control | 1,671.8 ± 549.2 | 325.1 ± 170.8 (100%) | 1.8 ± 0.6 |
| | (100%) | | |
| Preparation | 2,183.7 ± 170.3 | 373.8 ± 39.7 (99.5%) | 1.5 ± 0.3 |
| Example | (130.6%) | | |
| Comparative | 83.5 ± 17.7 | 5.6 ± 0.8 (2%) | 3.2 ± 1.9 |
| Preparation | (5%) | | |
| Example | | | |

*¹Area under the curve of blood concentration till 24 hrs
*²Maximum blood concentration
*³Time at the maximum blood concentration

As can be seen in Table 19 and FIG. 5, the formulation prepared in accordance with the present invention showed a higher bioavailability than prograf capsule (control formulation) in all respects. Therefore, the combined use of the substituted cyclodextrin and organic acid used in the present invention brings about a beneficial effect which was not achievable by previous arts.

While the invention has been described with respect to the above specific embodiments, it should be recognized that various modifications and changes may be made and also fall within the scope of the invention as defined by the claims that follow.

What is claimed is:

1. An amorphous taclolimus solid dispersion comprising taclolimus, a substituted α-, β- or γ-cyclodextrin derivative of formula (II) and an organic acid: 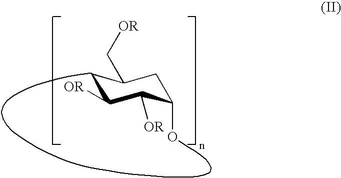
wherein,
n is an integer in the range from 6 to 8; and
R is C₁₋₆alkyl optionally substituted with hydroxyl, carboxy or carboxyC₁₋₄alkoxy, or sulfoC₁₋₄alkoxy.

2. The amorphous taclolimus solid dispersion of claim 1, wherein the weight ratio of taclolimus:substituted cyclodextrin derivative:organic acid ranges from 1:0.01˜120:0.1˜10.

3. The amorphous taclolimus solid dispersion of claim 1, which further comprises a surfactant, a water-soluble polymer or a pharmaceutically acceptable additive.

4. The amorphous taclolimus solid dispersion of claim 1, wherein the substituted cyclodextrin derivative is selected from the group consisting of 2-hydroxyethyl-α-cyclodextrin, 2-hydroxypropyl-β-cyclodextrin, 2,6-dimethyl-β-cyclodextrin, sulfobutylether-7-β-cyclodextrin, (2-carboxymethoxy)propyl-β-cyclodextrin, 2-hydroxyethyl-γ-cyclodextrin and 2-hydroxypropyl-γ-cyclodextrin.

5. The amorphous taclolimus solid dispersion of claim 4, wherein the substituted cyclodextrin derivative is selected from the group consisting of 2-hydroxyethyl-β-cyclodextrin, 2-hydroxypropyl-β-cyclodextrin, 2,6-dimethyl-β-cyclodextrin and sulfobutylether-7-β-cyclodextrin.

6. The amorphous taclolimus solid dispersion of claim 1, wherein the organic acid is selected from the group consisting of erythorbic acid, citric acid, tartaric acid, ascorbic acid, lactic acid, malic acid, succinic acid, acetic acid, trichloroacetic acid, trifluoroacetic acid, dimethyl triamine penta acetic acid, pyruvic acid, malonic acid, myristic acid, picric acid, methanesulfonic acid, ethanesulfonic acid, p-aminobenzoic acid, benzenesulfonic acid, benzoic acid, edetic acid, sorbic acid, adipic acid, gluconic acid, aminocapronic acid, glycyrrhizinic acid, isostearic acid, dodecyl benzenesulfonic acid, fumaric acid, maleic acid, oxalic acid, butyric acid, palmitic acid, sulfonic acid, sulfinic acid, formic acid, propionic acid, tannic acid, pantothenic acid, aspartic acid, aminoacetic acid, DL-a-aminopropionic acid and a mixture thereof.

7. The amorphous taclolimus solid dispersion of claim 6, wherein the organic acid is erythorbic acid or citric acid.

8. The amorphous taclolimus solid dispersion of claim 1, wherein the surfactant is selected from the group consisting of polyoxyethylene-sorbitan-fatty acid esters, sorbitan fatty acid esters, polyoxyethylene-polyoxypropylene block copolymer, reaction products of natural or hydrogenated vegetable oil with ethylene glycol, polyoxyethylene fatty acid esters, sodium dioctyl sulfosuccinate, sodium lauryl sulfate, a mixture of glycerol mono-, di- and tri-ester, polyethylene glycol mono- and di-ester, and polyethylene glycols.

9. The amorphous taclolimus solid dispersion of claim 3, wherein the water-soluble polymer is selected from the group consisting of alkyl cellulose, hydroxyalkyl cellulose, hydroxyalkylalkyl cellulose, carboxyalkyl cellulose and alkali metal thereof, carboxyalkylalkyl cellulose, carboxyalkyl cellulose ester, starch, pectin, chitin derivative, polysaccharides, polyacrylic acid and salt thereof, polymetacrylic acid and salt thereof, metacrylate copolymer, aminoalkyl metacrylate copolymer, polyvinyl acetal, diethyl aminoacetate, saccharic type surfactant, polyvinyl alcohol, polyvinyl pyrrolidone, polyvinyl pyrrolidone-vinyl acetate copolymer, polyalkylene oxide, and ethylene oxide and propylene oxide polymer.

10. A pharmaceutical composition comprising the amorphous taclolimus solid dispersion of claim 1, a surfactant, a water-soluble polymer and a pharmaceutically acceptable additive.

11. A method for the preparation of the amorphous taclolimus solid dispersion of claim 1, which comprises the steps of (1) dispersing or dissolving a substituted cyclodextrin derivative and an organic acid in an organic solvent; (2) dissolving taclolimus in an organic solvent; and (3) mixing the dispersion and solution obtained above, followed by removing solvents from the mixture by spray drying.

12. The method of claim 11, wherein the solvent used in step (2) is selected from the group consisting of ethanol, isopropyl alcohol, acetone, acetonitrile, dichloromethane, chloroform and a mixture thereof.

13. The method of claim 11, wherein a surfactant, a water-soluble polymer or a pharmaceutically acceptable additive is further added to the solution of step (1).
